SEQUENCE LISTING (1 sequence)

SEQ ID NO: 1 - protein, 9 aa, Homo sapiens
KLQCVDLHV

(19) United States
(12) Patent Application Publication
(10) Pub. No.: US 2008/0095790 A1
(43) Pub. Date: Apr. 24, 2008

(54) Methods of Treating Prostate Cancer

(76) Inventors: Supriya M. Perambakam, Chicago, IL (US); David J. Peace, Chicago, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/923,289

(22) Filed: Oct. 24, 2007

Related U.S. Application Data
(60) Provisional application No. 60/854,284, filed on Oct. 24, 2006.

(51) Int. Cl. A61K 39/00; A61P 35/00

(52) U.S. Cl. 424/185.1

(57) ABSTRACT

A method of treating prostate cancer in a human using a prostate-specific antigen peptide vaccine and a dosing regimen for administering said vaccine is provided.

### CROSS-REFERENCE TO RELATED APPLICATION

This application claims the benefit of prior U.S. provisional application No. 60/854,284 filed Oct. 24, 2006, hereby incorporated by reference in its entirety.

The invention was funded in part by U.S. government grants from National Institutes of Health (NIH) grant CA088062, and U.S. Army Medical Research and Material Command grant DAMD17-98-18489. The government has certain rights in the invention.

### FIELD OF THE INVENTION

The invention relates to methods of treating prostate cancer.

### BACKGROUND

Prostate cancer (CaP) is the most common malignancy (33% of all cancers) and the third leading cause of cancer related mortality (9% of cancer deaths) in men in the United States (Walczak and Carducci, (2007) Mayo Clin Proc. 82:243-249). Patients who present with locally advanced, unresectable tumors or frankly metastatic disease usually progress to hormone-refractory disease for which curative systemic therapies are lacking. Affected individuals often are elderly with limited tolerance for conventional cytotoxic chemotherapies. Thus, the need for effective, yet readily tolerated treatments is apparent.

A number of highly tissue-restricted proteins that are potential targets for attack by auto-reactive T-cells are expressed by CaP. Such proteins might serve as de facto tumor specific antigens, if they are targeted following conventional primary therapy, i.e. surgery or radiation of the prostate. One such marker, prostate specific antigen (PSA), is expressed on most of the prostate adenocarcinomas and therefore has received attention as a potential target for specific T-cell immunotherapy of CaP (Eder et al., (2000) Clin Cancer Res 6:1632-8; Meidenbauer et al., (2000) Prostate 43:88-100; Heiser et al., (2002) J Clin Invest 109:409-17; Barrou et al., (2004) Cancer Immunol Immunother 53:453-60; Gulley et al., (2005) Clin Cancer Res 11:3353-62).

PSA is a 34-kDa kallikrein-like serine protease, which is produced by ductal and acinar epithelial cells of the prostate gland in males (Li and Beling, (1973) Fertil Steril 24: 134; Watt et al., (1986) Proc Natl Acad Sci USA 83:3166). PSA is detected in most adenocarcinomas of the prostate, and hence may be a useful target antigen for T-cell immunotherapy (Ford et al., (1985) Br J Urol 57: 50). A nonameric segment of PSA (amino-acid position 146-154; sequence KLQCVDLHV, SEQ ID NO: 1) which binds to the prevalent human leukocyte antigen, HLA-A2, was discovered that elicits specific cytotoxic T lymphocyte (CTL) responses in normal individuals (Xue et al., (1997) Prostate 30:73) and in patients with prostate cancer of the HLA-A2 phenotype (Perambakam et al., (2002) Cancer Immunol Immunother 51: 263-270). It has been shown that CTL induced by immunization with dendritic cells (DC) pulsed with killed tumor cells that express PSA protein recognize target cells pulsed with the PSA 146-154 peptide (Nouri-Shirazi et al., (2000) J Immunol, 165: 3797). These findings imply that the PSA 146-154 peptide is naturally processed and presented by PSA+ tumors of the HLA-A2 phenotype. Importantly, homologous segments of other known members of the human kallikrein family do not share key HLA-A2 binding anchor residues of the PSA 146-154 sequence. These observations predict the usefulness of the PSA 146-154 peptide as a target antigen for T-cell immunotherapy of CaP.

In patients with CaP, similar to other human malignancies, the immune system retains the potential to recognize native self-determinants associated with tumor cells (Nair et al., (1999) Int J Cancer 82: 121). Prostate specific antigen (PSA) is a 34 kd kallikrein-like serine protease, which is exclusively produced by ductal and acinar epithelial cells of the prostate gland in males (Li and Beling, (1973) Fertil Steril 24: 134; Watt et al., (1986) Proc Natl Acad Sci USA 83:3166). Several lines of evidence support the possibility that PSA may be a useful target antigen for specific T-cell immunotherapy of CaP. Firstly, the expression of PSA is highly restricted to normal and transformed prostatic epithelial tissues. Secondly, PSA is expressed at substantial levels by most adenocarcinomas of the prostate. Thirdly, the potential danger of developing debilitating autoimmune injury to normal tissues is limited because the prostate gland is not essential for survival and is often removed as a part of conventional primary therapy.

Thus there exists a need in the art for methods of treating CaP which demonstrably stabilize or reduces symptoms associated with CaP.

### SUMMARY OF THE INVENTION

The present invention provides methods for treating prostate cancer in a human comprising the step of administering a prostate specific antigen (PSA) peptide in an amount effective to stabilize or reduce serum PSA levels in a treatment regimen comprising co-administration of said PSA peptide with granulocyte monocyte colony stimulating factor (GM-CSF) or a treatment regimen comprising administration of dendritic cells pulsed with said PSA peptide, said peptide consisting of the amino acid sequence set out in SEQ ID NO: 1. In embodiments of the invention wherein the method utilizes a PSA peptide and GM-CSF, in one aspect administration is intradermal.

In various aspects of the invention, the PSA peptide and GM-CSF are co-administered in a weight-to-weight ratio of about 1:5.

In still other aspects, the PSA peptide and GM-CSF are co-administered in a single dose.

In one aspect, the dendritic cells, whether autologous or heterologous, are expanded in culture for at least seven days prior to being pulsed with said PSA antigen.

In another aspect, peripheral blood mononuclear cells (PBMC) are stimulated with 20 μg/ml of PSA peptide.

The invention further provides for treatment of CaP which results in an increase in PSA peptide-tetramer staining CD8⁺ cells of at least two-fold.

Methods provided are amenable for use with individuals who have high risk, locally advanced disease, individuals that have metastatic, hormone sensitive disease, individuals who have undergone radiotherapy or surgical ablation prior to beginning the method of treatment, and/or individuals who have completed a minimum of six weeks of primary treatment prior to beginning the method of treatment.

### DESCRIPTION OF THE DRAWINGS

FIG. 1 depicts a comparison of fold increase in PSA-peptide-tetramer⁺ cells between the two vaccination methods.

FIG. 2 depicts the correlation between IL-4 expression and serum PSA progression.

FIG. 3 demonstrates that DTH derived T-cells exhibit peptide-specific cytokine responses.

FIG. 4 demonstrates that DTH derived T-cells exhibit specific cytolysis of PSA-peptide pulsed target cells.

FIG. 5 depicts the absolute serum PSA levels of patients at different study time points.

### DETAILED DESCRIPTION OF THE INVENTION

The present invention discloses the unexpected discovery that the use of a small PSA-peptide-based vaccine can be at least as effective as a vaccine utilizing the full length PSA recombinant protein. The immunogenic potential of PSA administered as a whole-protein or expressed in its entirety with DNA and RNA vectors has been evaluated in several phase 1 clinical trials (Sanda et al., (1999) Urology 53:260; Eder et al., (2000) Clin Cancer Res 6:1632-8; Meidenbauer et al., (2000) Prostate 43:88-100; Heiser et al., (2002) J Clin Invest 109:409-17; Barrou et al., (2004) Cancer Immunol Immunother 53:453-60; Gulley et al., (2005) Clin Cancer Res 11:3353-62; Mahadevan M et al., (2007) Cancer Immunol Immunother 56:1615-1624). Vaccination with defined peptide epitopes of PSA as demonstrated herein have proven advantageous compared to methods that use whole PSA protein. That is, the peptide-based treatment elicited more focused responses, and, thus, limits the risks of unexpected auto-immune injury. The invention provides the first demonstration showing the therapeutic efficacy of HLA-A2 binding, PSA146-154 peptide in humans. The present invention successfully demonstrates the induction of CD8+ T-cell responses in situ that correlated with clinical responses.

Accordingly, in one embodiment, methods are provided for treating prostate cancer comprising the step of administering a PSA peptide in an amount effective to stabilize or reduce serum PSA levels wherein the PSA peptide is administered with an agent having the biological activity of stimulating an immune response. Methods provided contemplate the use of any agent that stimulates, promotes or otherwise augments an immune response. In one embodiment, the agent is an adjuvant and/or a cytokine, and in various aspects, the cytokine is tumor necrosis factor, interleukin-2, interleukin-4, interleukin-12, granulocyte macrophage colony stimulating factor, γ-interferons an/or combinations thereof.

In another embodiment, the PSA peptide and GM-CSF are co-administered in a weight-to-weight ratio of about 1:5. Other contemplated ratios are about 1:6, about 1:7, about 1:8, about 1:9, or about 1:10.

In a further embodiment, the PSA peptide and GM-CSF are co-administered in 2, 3, 4, 5, 6, 7, 8, 9, 10, 11, 12, 13, 14, 15, 16, 17, 18, 19, 20, 25, 30, 35, 40, 45, or 50 doses.

In another embodiment, the invention provides administration of dendritic cells pulsed with said PSA peptide. In various aspects, the antigen-presenting cells are autologous to the recipient of the treatment or heterologous to the recipient of the treatment. In one aspect, the antigen-presenting cells are dendritic cells, whether autologous or heterologous, and are expanded in culture prior to being pulsed with the PSA peptide. The culture methods known in the art for expanding antigen-presenting cells are to be used in the practice of the invention. In alternative aspects, the dendritic cells are expanded in culture for at least about 8, about 9, about 10, about 11, about 12, about 13, about 14, about 15, about 16, about 17, about 18, about 19, about 20, or at least about 21 days prior to being pulsed with PSA antigen. In alternative aspects, the dendritic cells are pulsed with about 25 μg/ml, about 30 μg/ml, about 35 μg/ml, about 40 μg/ml, about 45 μg/ml, about 50 μg/ml, about 100 μg/ml, about 150 μg/ml, about 200 μg/ml, about 300 μg/ml, about 350 μg/ml, about 400 μg/ml, about 450 μg/ml or about 500 μg/ml.

Routes of administration, whether for the PSA peptide and an agent that stimulates an immune response, or an antigen-presenting cell pulsed with the PSA peptide, include intravenous, subcutaneous, intramuscular, intraperitoneal, intradermal, oral, intranasal, intradermal, and intrapulmonary (i.e., by aerosol). The chosen route of administration will dictate the formulation that is administered and would be understood by the clinician of skill in the art.

In various embodiments, treatment of CaP which results in an increase in PSA peptide-tetramer staining CD8⁺ cells (Kim et al., J Immunology, 2000, 165: 7285-7299) of at least about 10%, at least about 20%, at least about 30%, at least about 40%, at least about 50%, at least about 60%, at least about 70%, at least about 80%, at least about 90%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold or at least about 20-fold is contemplated.

Vaccine compositions, in general, comprise one or more antigens formulated, combined, mixed, incorporated into and/or matrixed with one or more adjuvants, diluents, carriers and the like that is administered to a subject by any suitable route to induce protective and/or ameliorative immune responses to the antigen. “Adjuvant” refers to any substance that is distinct from the antigen which when incorporated into a vaccine acts generally to accelerate, prolong, enhance, augment and/or potentiate the host's immune response to the antigen, and includes compositions encompassed by the terms immunomodulator, immunopotentiator and immunoenhancer. In general, adjuvants comprise a heterogeneous group of compounds broadly classified as oil emulsions, mineral compounds, bacterial products, liposomes and immunostimulating complexes (ISCOMs).

Exemplary adjuvants include without limitation, ADJUMER™ (polyphosphazene); aluminum phosphate gel; algal glucans; algammulin; aluminum hydroxide gel (alum); high protein adsorbency aluminum hydroxide gel; low viscosity aluminum hydroxide gel; AF or SPT (emulsion of squalane (5%), Tween 80 (0.2%), Pluronic L121 (1.25%), phosphate-buffered saline pH 7.4); AVRIDIE™ (propanediamine); BAY R1005™ ((N-(2-Deoxy-2-L-leucylamino-b-D-glucopyranosyl)-N-octadecyldod-ecanoylamide hydroacetate); CALCITRIOL™ (1α,25-dihydroxyvitamin D3); calcium phosphate gel; CAP™ (calcium phosphate nanoparticles); cholera holotoxin, cholera toxin A1-protein A-D fragment fusion protein, cholera toxin B subunit; CRL 1005 (Block Copolymer P1205); cytokine containing liposomes; DDA (dimethyldioctadecylammonium bromide); DHEA (dehydroepiandrosterone); DMPC (dimyristoyl phosphatidylcholine); DMPG (dimyristoyl phosphatidylglycerol); DOC/Alum Complex (Deoxycholic Acid Sodium Salt); Freund's Complete Adjuvant; Freund's Incomplete Adjuvant; Gamma Inulin; Gerbu Adjuvant (mixture of: i) N-Acetylglucosaminyl-(P1-4)-N-acetylmuramyl-L-alanyl-D-glutamine (GMDP), ii) Dimethyl dioctadecylammonium. chloride (DDA), iii) Zinc L-proline salt complex (ZnPro-8); GM-CSF; GMDP (N-acetylglucosaminyl-(b1-4)-N-acetylmuramyl-L-al-anyl-D-isoglutamine); IC31™; Imiquimod (1-(2-methypropyl)-1H-imidazo[4,5-c]quinol-in-4-amine); ImmTher™ (N-acetylglucosaminyl-N-acetyhnuramyl-L-Ala-D-iso-Glu-L-Ala-glycerol dipalmitate); DRVs (Immunoliposomes prepared from Dehydration-Rehyrdation Vesicles); Interferon-.gamma.; Interleukin-1.beta.; Interleukin-2; Interleukin-7; Interleukin-12; ISCOMS™ (Immune Stimulating Complexes); ISCOPREP 7.0.3.™; Liposomes; LOXORIBINE™ (7-allyl-8-oxoguanosine); LT Oral Adjuvant™ (E. coli labile enterotoxin protoxin); Microspheres and Microparticles of any composition; MF59™; (squalene.water emulsion); MONTANIDE ISA 51™ (purified Incomplete Freund's Adjuvant); MONTANIDE ISA 72™ (metabolizable oil adjuvant); MPL™(3-Q-desacyl-4′-monophosphoryl lipid A); MTP-PE and MTP-PE liposomes ((N-acetyl-L-alanyl-D-isoglutaminyl-1-L-alanine-2-(1,2-dipalmitoyl-sn-glycero-3-(hydroxy-phosphoryloxy)) ethylamide, mono sodium salt); MURAMETIDE™ (Nac-Mur-L-Ala-D-Gln-OCH3); MURAPALMITINE™ and D-MURAPALMITINE™ (Nac-Mur-L-Thr-D-isoGIn-sn-glycerol dipalmitoyl); NAGO (Neuraminidase-galactose oxidase); Nanospheres or Nanoparticles of any composition; NISVs (Non-Ionic Surfactant Vesicles); PLEURAN™ (.beta.-glucan); PLGA, PGA and PLA (homo- and co-polymers of lactic and glycolic acid; micro-/nanospheres); PLURONIC L121™; PMMA (polymethyl methacrylate); PODDS™ (oroteinoid microspheres); Polyethylene carbamate derivatives; Poly rA:Poly rU (Poly-adenylic acid-poly-uridylic acid complex); Polysorbate 80 (Tween 80); Protein Cochleates (Avanti Polar Lipids, Inc., Alabaster, Ala.); STIMULON™ (QS-21); Quil-A (Quil-A saponin); S-28463 (4-Amino-otec,-dimethyl-2-ethox-ymethyl-1H-imidazo[4,5-c]quinoline-1-ethanol); SAF-1™ (Syntex Adjuvant Formulation); Sendai proteoliposomes and Sendai-containing lipid matrices; Span-85 (sorbitan trioleate); Specol (emulstion of Marcol 52, Span 85 and Tween 85); Squalene or Robane® (2,6,10,15,19,23-hexamethyl-ltetracosane and 2,6,10,15,19,23-hexamethyl-2,6,10,14,18,22 tetracosahexaene); Stearyl Tyrosine (Octadecyl tyrosine hydrochloride); Theramide® (N-acetylglucosaminyl-N-acetylinuramyl-L-Ala-D-isoGlu-L-A1-a-dipalmitoxy propylamide); Theronyl-MDP (Termurtide™ or [thr 1]-MDP; N-acetyl muramyl-L-threonyl-D-isoglutamine); Ty Particles (Ty-VLPs or virus like particles); Walter Reed Liposomes (Liposomes containing lipid A adsorbed to aluminum hydroxide), and the like.

Methods are contemplated which include combination therapy with a chemotherapeutic agent. Chemotherapy treatment can employ anti-neoplastic agents including, for example, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

The invention is illustrated by the following examples, which are not intended to be limiting in any way.

### EXAMPLES

### Example 1

### Clinical Trial Using PSA 146-154

A total of 28 HLA-A2+ patients with pathologically confirmed CaP were enrolled in the phase1b clinical trial. All subjects were between 50 and 80 years of age at the time of enrollment. Informed consent was obtained from all patients with authorization of the Institutional Review Board of the University of Illinois. All patients had undergone radio-therapy or surgical ablation of the prostate and had completed primary therapy a minimum of 6 weeks prior to enrollment in the vaccine study. Fourteen patients had high-risk, locally advanced disease (Group A), and fourteen patients had metastatic, hormone sensitive disease (Group B). Group A—patients had either (i) T3, T4 disease (ii) serum PSA levels greater than 10 (ng/ml) or (iii) a Gleason grade of at least 7. Group B—patients had stage D disease following primary therapy, with a declining serum PSA, and/or a stable or improving bone scan or CT scan in response to hormone therapy. All patients were tested against an anergy panel of mumps, measles and Candida prior to vaccination and were found to be reactive. Table 1 below summarizes the information on the patients enrolled in the trial.

TABLE 1

| | Group A | | Group B | | Protocol-1 | | Protocol-2 | | Total |
|---|---|---|---|---|---|---|---|---|---|
| Characteristic | n = 14 | | n = 14 | | n = 14 | | n = 14 | | n = 28 |
| Age | | | | | | | | | |
| median (avg) | 61.5 | (62.2) | 62 | (64) | 64.5 | (65.2) | 60.5 | (61) | 62 |
| range | 51-73 | | 51-80 | | 51-80 | | 51-75 | | 51-80 |
| Race | | | | | | | | | |
| white | 10 | | 13 | | 12 | | 11 | | 23 |
| black | 2 | | 1 | | 1 | | 2 | | 3 |
| other | 2 | | 0 | | 1 | | 1 | | 2 |
| ECOG PS | | | | | | | | | |
| 0 or 1 | 14 | | 14 | | 14 | | 14 | | 28 |
| 2 or 3 | 0 | | 0 | | 0 | | 0 | | 0 |
| Disease status | | | | | | | | | |
| undetectable (PSA 0) | 3 | | 4 | | 5 | | 2 | | 7 |
| measurable | 0 | | 7 | | 2 | | 5 | | 7 |
| increased PSA only | 11 | | 3 | | 7 | | 7 | | 14 |
| Sites of disease | | | | | | | | | |
| bone | 0 | | 4 | | 2 | | 2 | | 4 |
| soft tissue | 0 | | 4 | | 0 | | 4 | | 4 |
| Family history | | | | | | | | | |
| positive | 4 | | 3 | | 5 | | 2 | | 8 |
| negative | 9 | | 9 | | 6 | | 12 | | 17 |
| unknown | 1 | | 1 | | 2 | | 0 | | 3 |
| Gleason Score | | | | | | | | | |
| median (avg) | 7 | (7.14) | 7 | (7.3) | 7 | (7.2) | 7.5 | (7.25) | 7 |
| range | 4-9 | | 5-10 | | 5-10 | | 4-9 | | 4-10 |
| PSA at diagnosis | | | | | | | | | |
| median (avg) | 5.8 | (9.1) | 15.25 | (28.4) | 8.4 | (12.3) | 13.4 | (24.3) | 10.5 |
| range | 4-23.4 | | 3.4-139 | | <4-23.4 | | 3.4-139 | | 3.4-139 |
| PSA at study entry | | | | | | | | | |
| median (avg) | 0.32 | (3.66) | 0.4 | (2.6) | 0.4 | (2.75) | 0.4 | (3.5) | 0.4 |

| | | | | | |
|---|---|---|---|---|---|
| range | 0-12.5 | 0-13.8 | 0-12.1 | 0-13.8 | 0-13.8 |
| Local Therapy | | | | | |
| RPE | 2 | 4 | 2 | 4 | 6 |
| RPE + EBRT | 7 | 4 | 8 | 3 | 11 |
| EBRT, primary | 2 | 4 | 2 | 4 | 6 |
| other | 3 | 2 | 2 | 3 | 5 |
| Hormone Rx | | | | | |
| none | 10 | 0 | 5 | 5 | 10 |
| first line | 4 | 11 | 8 | 7 | 15 |
| second line | 0 | 2 | 1 | 1 | 2 |
| ≧3 therapies | 0 | 1 | 0 | 1 | 1 |
| Basal biochemistry | | | | | |
| Alkaline Phosphatase | 48-90 | 49-105 | 49-90 | 50-105 | 48-105 |
| Haemoglobin | 12.8-16.6 | 11.5-16.9 | 11.5-15.7 | 12.2-16.9 | 11.5-16.9 |
| Creatinine | 0.7-1.3 | 0.8-1.2 | 0.8-1.3 | 0.7-1.3 | 0.7-1.3 |

Patients were randomly assigned to treatment protocol-1 or protocol-2. To ensure a balance in patient features among the 2 treatment arms, both group A and B (described earlier) patients were represented. Protocol-1: PSA-peptide (100 μg) and GM-CSF (500 μg) mixed in 33% clinical grade DMSO (Edwards Lifesciences, Irvine, Calif.) in a total volume of 1 ml were administered intradermally as five (0.2 ml aliquot) injections on the volar aspect of the right arm. Patients were immunized concurrently with the HLA-A2 binding Flu-M1 peptide in the same manner on the left arm at the same concentration. Protocol-2: Patients underwent 7-9 liter leukapheresis at the Blood Donor Center of the University of Illinois. Peripheral blood mononuclear cells (PBMC) were obtained by centrifugation over Ficoll-Hypaque (Amersham Biosciences, Uppsala, Sweden). DC were cultured from monocytes as per the method of Lau et al., (2001) J Immunother 24:66-78. Briefly, adherent monocytes were cultured in T-150 flasks (Corning, Big Flats, N.Y.) containing 25 ml AIM-V medium (Life Technologies, Grand Island, N.Y.), IL-4 (1000 U/ml) and GM-CSF (1000 U/ml) at 37° C. in a 5% CO2 incubator for 7 days. On day 7, PSA-peptide and Flu-M1 peptide (20 μg/ml of each peptide) were added and incubated further for 16-18 hours. On day 8, DC were harvested by gentle mechanical agitation, washed three times with Dulbecco's PBS (BioWhittaker, Walkersville, Md.) and then re-suspended in 50 ml of normal saline and aseptically transferred to an infusion bag (Baxter, Irvine, Calif.). DC were then irradiated at 2000 rads and aseptically transferred to freezing bags (Baxter, Irvine, Calif.) using a 10 cc syringe with a 19 gauge needle and cryopreserved in a freezing mixture containing 30% plasmalyte, 10% DMSO and 10% human serum albumin and stored in liquid N2. Release criteria for the final DC product included, sterile bacterial, fungal and mycoplasma cultures, negative endotoxin per Limulus Amoebacyte lysate assay, viability of at least 90% and greater than 50% CD86, CD80, HLA-DR or CD1a positive cells and less than 10% CD14 positive cells by flow cytometric analysis. At the time of infusion, DC were rapidly thawed at 37° C., again checked for sterility and viability and administered intravenously to patients. Dose of DC ranged from 0.94 to 2.02×10⁸ cells per vaccine (average 1.499, median 1.555). Patients were vaccinated on weeks 1, 4, and 10 and were monitored for 4 hours after every vaccination. One patient (UPIN88) received only a single vaccination as one aliquot of the vaccine product subsequently came back positive for Paecilomyces that precluded further vaccinations of this patient.

All toxicities were graded using the NIH Common Terminology Criteria for Adverse Events, version 3.0 (Table 2). Both the methods of vaccination were well tolerated by a great majority of patients. All 28 patients showed mild pain, itching, and erythema with or without induration at the injection site. A patient, UPIN 49, had ureteral obstruction due to significant intrapelvic tumor progression. UPIN2 developed progressive disease and a pulmonary embolus which was not considered to be related to the investigational treatment. One patient (UPIN 88) received a contaminated DC product possibly Paecilomyces without untoward consequences.

TABLE 2

| | | |
|---|---|---|
| | Grade 1 or 2 | Grade 3 or 4 |
| | Number of | Number of |
| Adverse Event | patients (%) | patients (%) |

| Protocol-1 (n = 14) | | | |
|---|---|---|---|
| ureteral obstruction | 0 | | 1 (7) |
| injection site reaction | 14 | (100) | 0 |
| flu-like syndrome | 4 | (29) | 0 |
| myalgia | 3 | (21) | 0 |
| atrial flutter | 2 | (14) | 0 |
| hyperpigmentation | 1 | (7) | 0 |
| fatigue | 1 | (7) | 0 |
| anemia | 1 | (7) | 0 |
| peripheral edema | 1 | (7) | 0 |
| elevated creatinine | 1 | (7) | 0 |
| cough | 1 | (7) | 0 |
| prostatis | 1 | (7) | 0 |
| Protocol-2 (n = 14) | | | |
| thrombosis | 0 | | 1 (7) |
| fatigue | 1 | (7) | 0 |
| generalized rash | 1 | (7) | 0 |
| ureteral obstruction | 1 | (7) | 0 |
| flu-like syndrome | 1 | (7) | 0 |
| contaminated infusion | 1 | (7) | 0 |

All toxicities were graded using the NIH Common Terminology Criteria for Adverse Events, version 3.0.

### Example 2

### PSA-Peptide-HLA-A2 Tetramer Analysis

Frozen PBMC obtained at baseline (1 to 3 weeks before vaccination), week 26 and week 52 were thawed, washed checked for viability and re-suspended in RPMI-1640 medium (BioWhittaker, Walkersville, Md.) containing 10% human AB serum (complete medium). 2×10⁶ PBMC were plated in 24 well plates (Nunc, Naperville, Ill.) and cultured and in complete media containing PSA-peptide (20 μg/ml) and IL-2 (20 U/ml) for 7±1 days (1 cycle). In some patients, T-cell cultures were also stimulated with HLA-A2 binding control peptide, Flu-M1. Spent medium was aspirated and replenished with complete medium plus IL-2 and re-stimulated with irradiated autologous PBMC pulsed with peptide for additional 2 cycles prior to tetramer and cytokine analysis.

Recovered T-cells (1×10⁶ per tube) were doubly stained with PSA 146-154 peptide-tetramer-PE (Immunomics, San Diego, Calif.) and CD8-FITC (BD Biosciences, San Diego, Calif.) at room temperature for 30 minutes in phosphate-buffered saline containing 0.5% para-formaldehyde (Sigma, St. Louis, Mo.). Cells were washed, re-suspended in buffer and analyzed by a Calibure flow cytometer (Becton Dickinson, Mountain View, Calif.). Cells were gated on side-scatter (SSC) and forward-scatter (FSC) to include lymphocyte population. The percentage of PSA-peptide tetramer⁺CD8⁺ cells and PSA-peptide tetramer⁻CD8⁺ cells were determined from the quadrant dot plots using the Cell Quest software analysis.

Cells were also stained separately with negative Tetramer-PE, of unknown sequence that does not recognize CD8⁺ T-cells of any HLA allele to assess the level of background PE fluorescence and to appropriately set regions on the quadrant dot plots. As a positive control, Tetramer-PE staining for Flu-M1 peptide was also performed in some of the patients. The results are represented as the frequency of tetramer⁺ cells per CD8⁺ cells and is calculated as the number of tetramer⁺CD8⁺ cells divided by total number of CD8⁺ cells (tetramer⁺CD8⁺ cells and tetramer⁻CD8⁺ cells). A strong tetramer response is defined as >4-fold increase in tetramer frequency at week 26 and/or 52 compared to baseline.

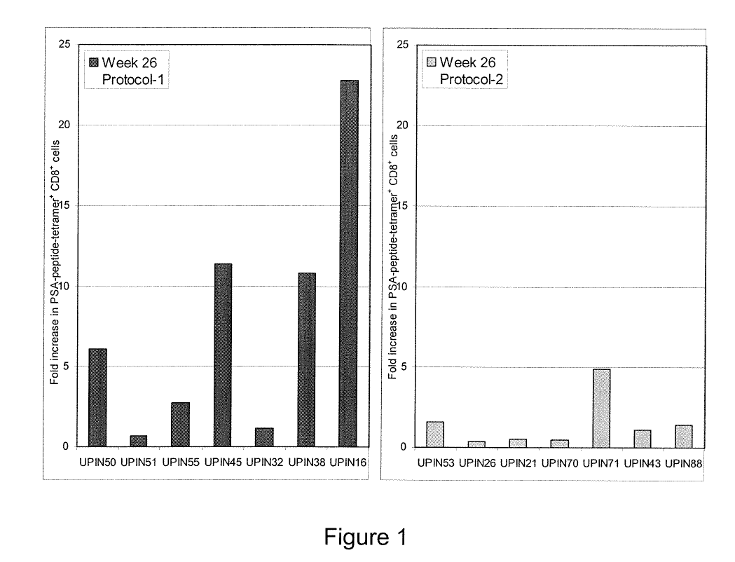

A total of 14 (50%) patients developed strong tetramer responses at week 26 and/or week 52. The average tetramer frequency of protocol-1 increased from baseline levels of 0.00732% to post-vaccine levels of 0.028787% and 0.019678% at weeks 26 and 52, respectively. Similarly, tetramer frequency of protocol-2 increased from baseline levels of 0.014315% to 0.041219% at week 26 and 0.021091% at week 52 (FIG. 1). At week 26 the fold increase in average tetramer frequency of protocol-1 was 4.4 times higher as compared to that of protocol-2 in the locally advanced group (p-value=0.007). However, no significant difference remained at week 52.

Tetramers combine 4 identical MHC/peptide complexes via a fluorescently-labeled biotin-avidin complex and have high avidity for T cell receptors (TCR). Tetramers can detect very rare, low frequency but highly specific T-cells and is therefore an important tool to quantify cellular immune responses ex vivo and to evaluate vaccine efficacy. The data indicated that 14 out 28 patients (50%) developed ≧4 fold increases in CD8⁺ PSA-peptide specific tetramer cells at week 26 and/or week 52. In a similar study, 9 of 25 (36%) of melanoma patients developed a 2 fold increase in tetramer positive cells following vaccination with HLA-A2⁺ binding MART-1, gp 100 and tyrosinase peptides (Markovic et al., (2006) Am J Clin Oncol. August; 29(4):352-60). Further, induction of PSA 146-154 peptide specific Tetramer positive T-cells correlated with reduced PSA progression.

At week 52, a total of 18 patients showed weak tetramer responses (<4 fold increase over baseline) of whom 11 (61%) evidenced PSA progression. By contrast, of the 9 patients who showed strong tetramer responses (≧4-fold increase over baseline) at week 52, only 3 (33%) had PSA progression. One of the three progressing patients (UPIN13) with strong tetramer responses subsequently experienced a sustained decline of serum PSA without further intervention (Table 3). He remains in good health with low serum PSA levels after more than four years post vaccination. Therefore, patients with negative tetramer responses at week 52 appeared to be more likely than positive responders to have progressive disease based on serum PSA levels. However the data is not statistically significant in this small cohort.

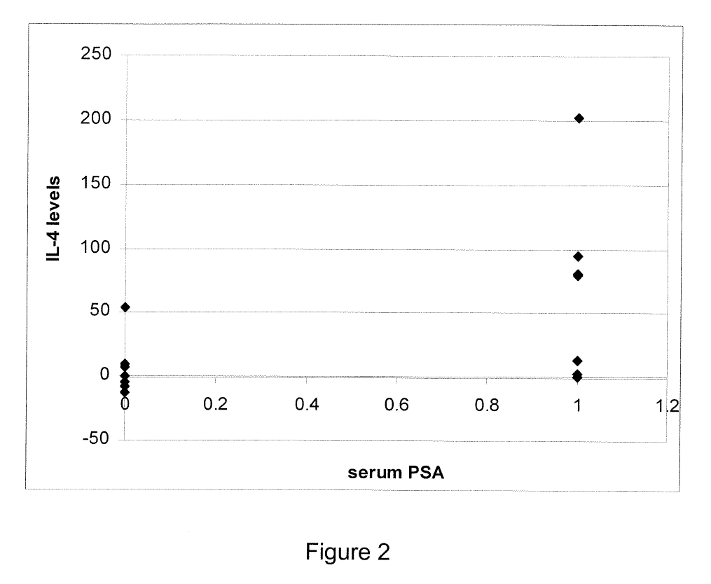

Patients who showed serum PSA progression had on an average higher IL-4 values at week 52 than subjects who did not show PSA progression (p=0.04). Therefore, higher expression of type 2 cytokine, IL-4, is probably associated with serum PSA progression (FIG. 2). IL-4 levels on the y-axis correspond to IL-4 values (μg/ml) obtained at week 52 minus the baseline levels. “0” on the x-axis denotes “non progression”, while “1” represents progression. Non progression is defined as less than 20% increase in serum PSA over baseline with an absolute PSA value less than 0.2 ng/ml and progression is defined as at least a 20% increase in serum PSA at week 52 over baseline with an absolute PSA value above 0.2 ng/ml. Patients who showed serum PSA progression had on an average higher IL-4 values at week 52 than patients who showed non progression per Spearman analysis (p=0.04).

Disease or clinical status of patients was evaluated per absolute serum PSA levels and bone/CT scans on weeks 1, 4, 7, 14, 26, and 52. Disease progression (P) is defined as at least a 20% increase in serum PSA at week 52 over baseline with an absolute PSA value above 0.2 ng/ml. Stable disease or non progression (NP) is defined as less than 20% increase in serum PSA over baseline or an absolute PSA value less than 0.2 ng/ml.

TABLE 3

| | Group | Fold increase in tetramer⁺ T- | | |
|---|---|---|---|---|
| | and | cells over baseline | | Clinical |
| Patient | Protocol | Week 26 | Week 52 | Status |
| UPIN50 | A1 | 6.115201 | 3.802251 | P |
| UPIN51 | A1 | 0.694325 | 0.26312 | P |
| UPIN55 | A1 | 2.713499 | 5.283747 | NP |
| UPIN45 | A1 | 11.3919 | 6.975612 | NP |
| UPIN32 | A1 | 1.139831 | 1.058785 | P |
| UPIN38 | A1 | 10.79409 | 1.370783 | P |
| UPIN16 | A1 | 22.77982 | 121.2959 | NP |
| UPIN13 | B1 | 22.25 | 34.4925 | P |
| UPIN37 | B1 | 0.809958 | 0.260138 | P |
| UPIN40 | B1 | 15.85366 | 2.477878 | NP |
| UPIN28 | B1 | 29.25345 | 12.50256 | NP |
| UPIN67 | B1 | 1.722796 | 0.071933 | NP |
| UPIN85 | B1 | 2.506368 | 0.08353 | P |
| UPIN49 | B1 | 1.755686 | 1.711501 | P |
| UPIN53 | A2 | 1.586671 | 0.691201 | NP |
| UPIN26 | A2 | 0.355337 | 0.816376 | NP |
| UPIN21 | A2 | 0.51283 | 5.432181 | NP |
| UPIN70 | A2 | 0.462146 | 1.85008 | P |
| UPIN71 | A2 | 4.9104 | 4.8272 | NP |
| UPIN43 | A2 | 1.118662 | 5.065763 | P |
| UP1N88 | A2 | 1.420318 | 0.0689 | P |
| UPIN2 | B2 | 3.096546 | 5.830912 | P |
| UPIN35 | B2 | 1.311615 | 0.091439 | P |
| UPIN69 | B2 | 2.099828 | 0.23888 | P |
| UPIN27 | B2 | 82.1169 | lfu | * |
| UPIN81 | B2 | 1.72969 | 0.096654 | NP |
| UPIN82 | B2 | 4.477975 | 0.242953 | P |
| UPIN89 | B2 | 2.934475 | 0.227472 | NP |
| *Disease progression (P) is defined as at least a 20% increase in serum PSA at week 52 over baseline or an absolute PSA value above 0.2 ng/ml. Stable disease or non progression (NP) is defined as less than 20% increase in serum PSA over baseline or an | | | | |
| # absolute PSA value less than 0.2 ng/ml. UPIN27 was lost to follow up (lfu) at week 52 and so his clinical status remains undetermined. A positive tetramer response is defined as ≧ 4-fold increase in tetramer frequency at week 26 and/or 52 compared to baseline. | | | | |

### Example 3

### Specific DTH Responses Induced by Vaccination with PSA-Peptide

A total of 28 patients were vaccinated with PSA-peptide at three time points, i.e., weeks 1, 4 and 10 and DTH skin testing was performed on weeks 4, 14, 26 and 52. Increasing doses of peptide from 1 to 20 μg elicited increasing level of DTH induration in responding patients. Injection of carrier only, i.e. 33% DMSO did not elicit significant induration.

Patients with both locally advanced and metastatic CaP responded to the vaccination. Fifty percent of the patients (14 of 28) developed positive DTH responses to PSA-peptide over time. In the remaining patients specific DTH responses were not observed and remained negative up to week 52.

In one patient, UPIN69, a strong DTH reaction to the PSA-peptide was observed at week 4 and was maintained over the course of the study. In the other 13 patients with positive DTH reactions no response was detected at initial testing on week 4. Specific responses became evident at week 14 or later and increased progressively with subsequent DTH testing indicating the induction of specific T-cell immunity. In one patient, UPIN49, a positive DTH reaction did not become evident until week 52. Thus, multiple peptide vaccinations appeared to augment the strength of DTH responses over time.

Phenotype of T-Cells Recovered from DTH Sites

Skin biopsy of the DTH site was obtained from 7 positive patients and provided the basis for the following data. T-cells were recovered from the dermal tissue using the CD3/CD28 micro-bead technique. Out-growth of T-cells was observed within 3 days and wells of the culture plates became confluent by 6-8 days. Recovered T-cells were phenotyped for CD4 and CD8 markers directly (cycle 0) or after stimulation with irradiated autologous peptide-pulsed PBMC supplemented with low dose IL-2 (20 U/ml) for 1-4 additional cycles.

Variable proportions of CD4⁺ CD8⁻, CD4⁻CD8⁺ and CD4⁺ CD8⁺ T-cells were recovered per flow cytometric analysis. The number of CD4⁻CD8⁺ T-cells was greater than CD4⁺ CD8⁻ T-cells in 4 of 7 patients at cycles 0, i.e. without peptide priming in vitro. Following in vitro stimulation with PSA-peptide, the number of CD4⁻CD8⁺ T-cells became greater than CD4⁺ CD8⁻ T-cells in 6 of 7 patients. However, in one patient, UPIN45, CD4⁺ CD8⁻ T-cells were higher than CD4⁻CD8⁺ T-cells. It is of interest to note that a significant percentage of CD4⁺ CD8⁺ T-cells (11-16%) was seen in one patient, UPIN 55, and these cells were consistently observed from cycle 0 through cycle 4 of stimulation. Attempts were made to isolate CD4⁺ CD8⁺ T-cells for further analysis, but sufficient cell numbers could not be recovered.

Cytokine Profile of T-Cells Derived from DTH Sites

T-cells recovered from skin biopsies of DTH-positive patients were examined to determine whether specific T-cells develop in situ. Sufficient T-cells were procured from one patient, UPIN55 for direct analysis at cycle 0, i.e. without peptide priming in vitro. T-cells exhibited specific cytokine responses against T2 cells pulsed with PSA-peptide compared to T2 cells pulsed with irrelevant HLA-A2 binding peptide, HIV-RT476-484, or T2 cells pulsed without peptide per CBA analysis.

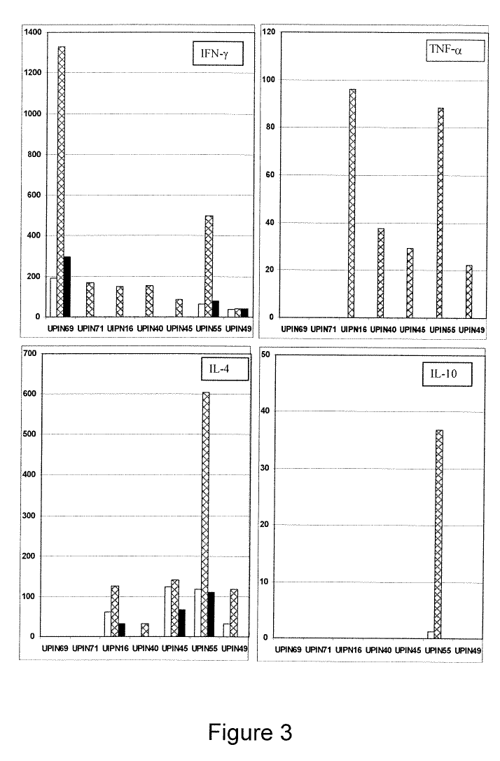

The cytokine profile following in vitro stimulation with PSA-peptide for 1-3 cycles was studied in all 7 patients. As shown in FIG. 3, T-cells derived from 6 out of 7 patients showed specific IFN-γ responses (85-1032 μg/ml, median 163). Specific levels of TNF-α (23-96 μg/ml, median 38) and IL-4 (18-486 μg/ml, 63) were observed for four patients, while IL-10 was detected in only one patient (37 μg/ml). Non-specific expression of IL-6 was seen in one patient (UPIN 49).

A skin biopsy of a DTH reaction to the FluM-1 peptide, a positive control peptide, was also available for one patient, UPIN69. Flu-M1-peptide specific T-cells generated IFN-γ levels (1090 μg/ml) that were comparable to that produced by PSA-peptide specific T-cells (1032 μg/ml).

DTH reactions to immunizing peptides have been observed in other peptide-vaccination protocols, particularly melanoma vaccine studies and are often used as an indicator of anti-tumor immunity and vaccine efficacy (Waanders et al., (1997) Clin. Cancer Res. 3:685; Nestle et al., (1998) Nat. Med. 4:328; Disis et al., (2000) Clin Cancer Res 6:1347). Thirteen of fourteen patients with positive DTH reactions showed no response when initially tested at week 4. Specific responses became evident at week 14 or later and increased progressively during subsequent DTH testing, indicating the induction of specific T-cell immunity. Although injection of PSA-peptide for DTH skin testing might immunologically resemble another round of vaccination, low doses (1-20 μg versus 100 μg) of naked peptide may not have induced that big an impact. Multiple cycles of peptide vaccination therefore appeared to augment the recruitment of specific T-cells to the DTH sites over time.

Direct recovery of a large number of T-cells from small clinical specimens, such punch biopsy of the skin, is often very difficult with traditional culture techniques involving only IL-2. Therefore, dermal tissues were cultured in the presence of CD3/CD28 micro-beads and low dose IL-2 for 6-8 days. T-cells were tested directly or following further stimulation with PSA-peptide in vitro. The expanded T-cells exhibited specificity to PSA-peptide per CRA and CBA analysis presumably reflecting corresponding responder T-cells that prevail in vivo, although, the actual frequency of specific T-cells are undoubtedly distorted by the procedure of in vitro stimulation.

The dermal tissue was minced into small fragments and co-cultured in RPMI-1640 medium (BioWhittaker, Walkersville, Md.) containing 10% human AB serum (complete medium) and IL-2 (100 U/ml) along with CD3/CD28 beads (Dynal, Oslo, Norway) at a concentration of 2.5×10⁵ per well in 1 ml of medium in 48 well plates (Nunc, Naperville, Ill.). Out-growth of T-cells was observed within 3 days. On day 4, 500 μl of the spent medium was aspirated and replenished with fresh medium plus IL-2 and cultured further for a total of 6-8 days, until the culture wells became confluent. Recovered T-cells were tested directly (cycle 0) or after 1-4 cycles of in vitro stimulation with irradiated autologous peptide-pulsed PBMC plus low dose IL-2 (20 U/ml). Each cycle of stimulation was 7±1 days.

Cytokines released into the culture supernatant, including IFN-γ, TNF-α, IL-4, IL-6 and IL-10, were measured concurrently by cytokine bead array (CBA) analysis (BD Biosciences, San Diego, Calif.). The antigen presenting cell line, T2 (ATCC, Manassas, Va.) was used as a stimulator and was pulsed with 20 μg/ml of PSA-peptide or control HLA-A2 binding peptide, HIV-RT476-484 or diluent alone (0.4% volume by volume). T2 cells (25,000/well) were cultured with DTH-derived T-cells (100,000/well) in complete medium containing 30 U/ml of IL-2 in a total volume of 1 ml per well in 48-well plates. This particular stimulator to responder ratio was found to be optimal for culture in 48-well plates. Cells were incubated at 37° C. for 24 hours in 5% CO2 atmosphere. Supernatants were harvested and stored in sterile vials at −80° C. At the time of assay, samples were thawed and cytokines were measured using a CBA kit as per the manufacturer's protocol with a FACSCalibur flow cytometer (Becton Dickinson, Mountain View, Calif.).

The cytokine profile following in vitro stimulation with PSA-peptide for 1-3 cycles was studied in all 7 patients. As shown in FIG. 3, T-cells derived from 6 out of 7 patients showed specific IFN-γ responses (85-1032 μg/ml, median 163). Specific levels of TNF-α (23-96 μg/ml, median 38) and IL-4 (18-486 μg/ml, 63) were observed for four patients, while IL-10 was detected in only one patient (37 μg/ml). Non-specific expression of IL-6 was seen in one patient (UPIN 49).

A skin biopsy of a DTH reaction to the FluM-1 peptide, a positive control peptide, was also available for one patient, UPIN69. Flu-M1-peptide specific T-cells generated IFN-γ levels (1090 μg/ml) that were comparable to that produced by PSA-peptide specific T-cells (1032 μg/ml).

Interestingly, the method of vaccination appeared to impact the pattern of cytokine responses to the PSA-peptide. DTH-derived T-cells, from two patients who exhibited only IFN-γ responses were vaccinated by protocol 2. On the other hand, five out of seven patients who exhibited multiple cytokine responses (see FIG. 3) were vaccinated by protocol 1.

### Example 4

### Specific Cytotoxicity of T-Cells Derived from DTH Sites

DTH derived T-cells were phenotyped for CD4 and CD8 markers. Briefly, 1×10⁵ cells were labeled with CD4-PE and CD8 FITC and gated on side-scatter (SSC) and forward-scatter (FSC) to include the lymphocyte population. Propidium iodide (Sigma, St. Louis, Mo.) was added (1 g/ml, final concentration) to each sample to exclude dead cells. All incubations were carried out at 4° C. for 15 minutes in phosphate-buffered saline containing 2% normal mouse serum and 0.01% sodium azide (Sigma, St. Louis, Mo.). The relative log fluorescence of viable cells was measured at 495 nm and the percentage of CD4⁺ and CD8⁺ T-cells were quantified using the Calibur. MIgG1-FITC and MIgG2a-PE served as isotype controls. All antibodies and isotype controls were purchased from PharMingen (San Diego, Calif.). In a separate experiment, CD4⁻CD8⁺ T-cells were sorted and collected aseptically using a Vantage flow cytometer (Becton Dickinson, Mountain View, Calif.).

Chromium Release Assay (CRA)

Specific cytolytic activity was analyzed by standard 4 hour CRA as previously described (Xue et al., (1997) Prostate 30:73). Briefly, 1×10⁶ T2 cells, a peptide transport-deficient B-lymphoblastoid x T-lymphoblastoid cell line (targets) were labeled with 100 μCi of Na⁵¹CrO₃ (Amersham Pharmacia Biotech, Piscataway, N.J.) and then pulsed with PSA-peptide or HIV-RT476-484 control peptide (Research Genetics, Huntsville, Ala.) or no peptide. Sorted CD4⁻CD8⁺ T-cells derived from positive DTH reaction site (effector cells) were plated in 96 well round-bottom plates (Nunc, Naperville, Ill.) in triplicates at indicated effector to target ratios and incubated with 1×10³ target cells per well for 4 hours. Supernatants recovered from CRA were assayed for gamma emission using a Top-count NXT scintillation counter (Packard, Meriden, Conn.) and the percent specific lysis was calculated as previously described (Xue et al., (1997) Prostate 30:73).

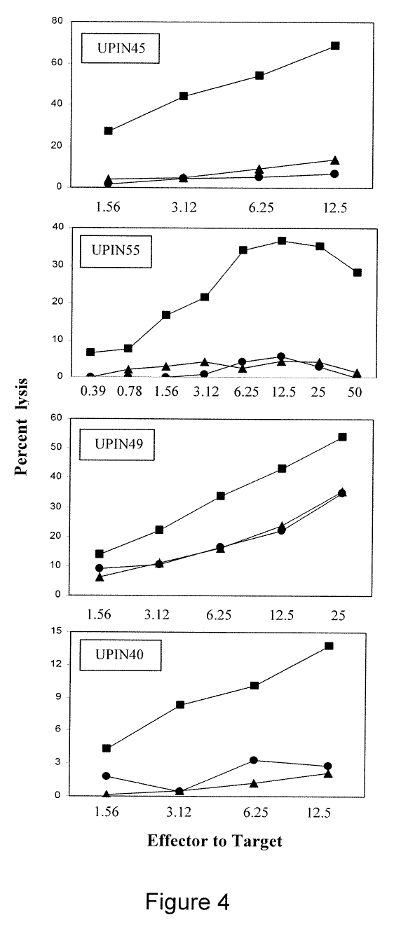

CRA were performed to determine whether T-cells derived from positive DTH reaction sites also exhibited specific cytotoxicity. Following the culture with CD3/CD28 beads, recovered T-cells were further stimulated in vitro with irradiated autologous PBMC pulsed with PSA-peptide for 1-4 cycles. Since flow cytometric analysis revealed mixed phenotypic populations (see Table IV), CD4⁻CD8⁺ T-cells were FACS sorted in 4 patients. Sorted CD4⁻CD8⁺ T-cells showed peptide-specific lysis of PSA-peptide-pulsed T2 cells compared to T2 cells pulsed with irrelevant HLA-A2 binding peptide, HIV-RT476-484, or T2 cells pulsed without peptide. As shown in FIG. 4, T-cells induced from patients UPIN45, UPIN 55, UPIN 49 were strongly cytolytic while T-cells from patient UPIN40 demonstrated modest cytolytic activity.

TABLE IV

| | Cycle of | % Positive cells | | |
|---|---|---|---|---|
| Patient | culture | CD4⁺CD8⁻ | CD4⁻CD8⁺ | CD4⁺CD8⁺ |
| UPIN16 | 0 | 32 | 62 | 8 |
| | 3 | 20 | 75 | 3 |
| UPIN40 | 0 | 39 | 48 | 10 |
| | 2 | 21 | 69 | 6 |
| UPIN45 | 0 | 78 | 12 | 7 |
| | 3 | 56 | 26 | 7 |
| UPIN49 | 0 | 46 | 27 | 6 |
| | 2 | 3 | 80 | 4 |
| UPIN55 | 0 | 50 | 29 | 13 |
| | 2 | 9 | 59 | 16 |
| | 4 | 12 | 65 | 11 |
| UPIN69 | 0 | 19 | 53 | 2 |
| UPIN71 | 0 | 39 | 46 | 6 |
| | 3 | 14 | 69 | 2 |
| Skin biopsies from 7 DTH positive patients were available for in vitro analysis. Lymphocytes were isolated from biopsies by co-culturing the tissue in medium along with CD3/CD28 beads for 6-8 days (cycle 0). Additionally | | | | |
| # T-cells were stimulated with PSA-peptide for 1-4 cycles. Each in vitro culture cycle consisted of 7 ± 1 days. 1 × 10⁵ cells were labeled with CD4-PE and CD8 FITC and gated on side-scatter and forward-scatter to include lymphocyte population. Percent positive cells were quantified by 2-color staining. | | | | |

### Example 5

### Clinical Outcomes

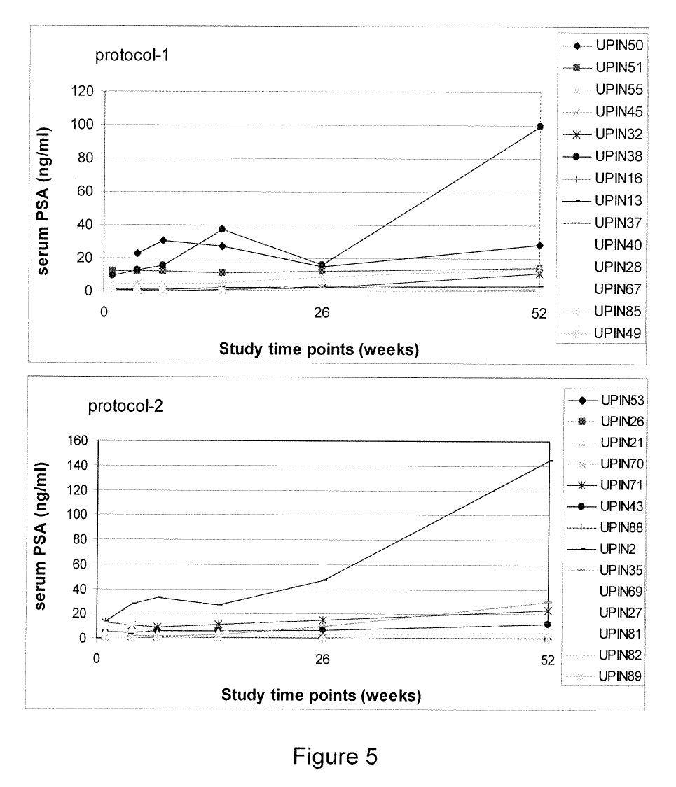

Disease status was evaluated by absolute serum PSA levels and bone/CT scans on weeks 1, 4, 7, 14, 26, and 52. Patients who showed at least a 20% increase in serum PSA levels over two consecutive time points or having a serum PSA values higher than 0.2 ng/ml were considered to have negative biochemical response and vice versa. Overall, 44% of the patients showed stable serum levels (6 patients in protocol-1 and 6 in protocol-2, and 1 patient in protocol 2 was lost to follow-up at week 52) within a follow-up period of 1 year (FIG. 5).

From the foregoing it will be appreciated that, although specific embodiments of the invention have been described herein for purposes of illustration, various modifications may be made without deviating from the spirit and scope of the invention.

What is claimed is:

1. A method for treating prostate cancer in a human comprising the step of administering a prostate specific antigen (PSA) peptide in an amount effective to stabilize or reduce serum PSA levels in a treatment regimen comprising co-administration of said PSA peptide with granulocyte monocyte colony stimulating factor (GM-CSF) or a treatment regimen comprising administration of dendritic cells pulsed with said PSA peptide, said peptide consisting of the amino acid sequence set out in SEQ ID NO: 1.

2. The method of claim 1 wherein co-administration of said PSA peptide and GM-CSF is intradermal.

3. The method of claim 1 wherein said PSA peptide and GM-CSF are co-administered in a weight-to-weight ratio of about 1:5.

4. The method of claim 1 wherein PSA peptide and GM-CSF are co-administered in multiple injections.

5. The method of claim 4 wherein PSA peptide and GM-CSF are co-administered in up to five injections.

6. The method of claim 4 wherein a total of about 100 μg PSA peptide is administered in multiple injections.

7. The method of claim 1 wherein administration of said dendritic cells pulsed with said PSA peptide is intravenous.

8. The method of claim 1 wherein said dendritic cells are autologous to said human being treated.

9. The method of claim 8 wherein said autologous dendritic cells are expanded in culture prior to being pulsed with said PSA peptide.

10. The method of claim 9 wherein said autologous dendritic cells are expanded in culture for at least seven days prior to being pulsed with said PSA antigen.

11. The method of claim 1 wherein said dendritic cells are stimulated with 20 μg/ml PSA antigen.

12. The method of claim 1 which results in an increase in PSA peptide-tetramer levels in CD8⁺ cells of at least two-fold.

13. The method of claim 1 wherein said human has high risk, locally advanced disease.

14. The method of claim 1 wherein said human has metastatic, hormone sensitive disease.

15. The method of claim 1 wherein said human has undergone radiotherapy or surgical ablation prior to beginning said method of treatment.

16. The method of claim 1 wherein said human has completed a minimum of six weeks of primary treatment prior to beginning said method of treatment.
